(19) United States
(12) Patent Application Publication
(10) Pub. No.: US 2005/0118212 A1
(43) Pub. Date: Jun. 2, 2005

(54) Lipid-bearing cosmetic preparation

(76) Inventor: Susanne Emig, Eckental (DE)

Correspondence Address: BACHMAN & LAPOINTE, P.C., 900 CHAPEL STREET, SUITE 1201, NEW HAVEN, CT, 06510, US

(21) Appl. No.: 10/505,814

(22) PCT Filed: May 9, 2003
(86) PCT No.: PCT/EP03/04883

(30) Foreign Application Priority Data
May 10, 2002 (DE) 202073289

(51) Int. Cl. A61K 7/42; A61K 7/021; A61K 7/06; A61K 7/11

(52) U.S. Cl. 424/401; 424/59; 424/63; 424/70.11; 424/74

(57) ABSTRACT

A lipid-bearing preparation, in particular in the form of a workable paste which is suitable for cosmetic uses, in particular in the region of decorative cosmetics, for coloring and improving the appearance of the skin, lips and eyelids. It is suitable, with suitable additives, also as lip gloss, as fixing means for lipstick, as lip care, as a skin care foundation or as a sun protection agent. That lipid-bearing preparation occurs in water-free form. It is free of waxes, lanolin and lanolin derivatives. It comprises the mixture of at least two oils, namely a straight long-chain ester and liquid polybutene which are put into a pasty form by the addition of hydrogenated triglycerides and finely divided solid substances such as silica, bentonite or hectorite. The preparation may additionally contain coloring agents and suitable cosmetic active substances. Processes for the production of that preparation are also described.

### BACKGROUND OF THE INVENTION

The invention concerns a lipid-bearing preparation, in particular in the form of a workable paste which is suitable for cosmetic uses, in particular in the region of decorative cosmetics, for coloring and improving the appearance of the skin, lips and eyelids. By way of example mention may be made here of lip rouge, blusher. makeup or eyeshadow. It can also be used as lip gloss, as a fixing agent for the lips, as a skin care foundation or as a sun protection agent. That lipid-bearing preparation occurs in particular in water-free form.

Preparations of the specified kind usually contain lipids such as for example fats, oils, oil-soluble vegetable extracts and medium to long-chain fatty acids and waxes.

In addition they may contain a solid phase comprising finely divided fillers and coloring agents. Sun protection agents may use particularly finely divided pigments, so-called nanopigments, of an average particle size of between 5 and 25 nm, which act transparently on the skin and no longer color it. Silicon dioxide, titanium dioxide and zinc oxide may be mentioned here by way of example.

A disadvantage with such preparations is that they can be transferred from the skin or the lips to which they are regularly applied on to other surfaces, for example cups, glasses, textiles or other areas of the skin. That can leave behind traces in the form of a colored mark or a greasy film. Such products therefore have insufficient adhesion to the underlying surface to which they are applied, with the result that lipstick and blusher, make up, eyeshadow and also sun protection agents have to be regularly re-applied. As oily constituents generally spread very well on the skin, the pigments move from the original place of application together with small amounts from the oily phase into the fine wrinkles and creases in the skin in the immediate surroundings, which has a very disturbing effect on the overall visual impression.

In the past the attempt was made to counteract that in relation to lipsticks and lip rouge by the use of so-called ‘bromo acids’, dyestuffs which substantively gather on the skin. As however, due to differing pH-values of the skin, those dyestuffs gave individually different and unpredictable shades and in addition the coloring effects often persisted for days, that route was immediately abandoned again.

After silicone oils and silicone resins had found their way into cosmetics, the attempt was made to improve the adhesion to the skin and thus the durability of decorative preparations thereby. Thus from about 1977 eyeshadow pencils and lipsticks have been known in the form of leads which were cast into injectable casings which in the lipid phase contained inter alia a mixture of phenyl trimethicone (a non-volatile silicone oil) and cyclomethicone (a volatile silicone oil). They were then followed by similar preparations which contained cyclomethicone as the sole silicone component. Although they had an apparently firm structure, those pencil materials could be applied to the skin in a soft and malleable fashion, similarly to a pasty material. After evaporation of the volatile silicone, there was left behind on the skin a soft elastic film which had very good adhesion and which moved only to a minimum extent into the area surrounding the original location at which it was applied. The principle of combining two silicone oils, applied to pasty preparations, is also to be found again for example in EP 0 756 864.

In spite of the highly positive effects in terms of adhesion and durability, such preparations which contain silicone oil can give rise to detrimental effects on the part of sensitive users if they are used in the immediate proximity of the eye. More specifically, if even minimum amounts of silicone oils pass into the eye or the conjunctiva sac, they can lead to an oily film on the lens and unpleasant irritation, referred to as the ‘wind burn effect’.

Therefore the object of the invention was to provide a lipid-bearing preparation, in particular in the form of a workable paste, which is suitable for cosmetic uses, in particular in the region of decorative cosmetics, for coloring and improving the appearance of the skin, the lips and the eyelids, which can be easily applied, which has good adhesion and which persists for a long time, and which travels from the original place of application into the immediate surroundings not at all or only to a minimal extent. The invention further seeks to provide that the preparation is substantially free of silicone oils or silicone derivatives in order to avoid the disadvantages thereof in the immediate region of the eye. The invention further seeks to provide that the preparation is stable in respect of storage at different storage temperatures which can occur on the different transportation routes and on the part of the users themselves, and in particular do not exhibit any syneresis effects after prolonged storage. In addition the preparation is to involve a viscosity in the range of between 1.5 and 12 Pas which does not substantially change even over a relatively long storage time. Here, the term storage time is used to denote the period of time between delivery to the trade and consumption on the part of the customer. Therefore, in comparison with products in the state of the art, that preparation is to be such that it can be applied softly and malleably, it does not become tight on the lips and does not dry them out, it has good and durable adhesion to the skin, as far as possible it does not transfer on to articles and textiles or other regions of the skin and in the immediate vicinity of the eye it does not give rise to irritation.

### SUMMARY OF THE INVENTION

The object is achieved by providing a preparation which preferably contains no waxes, no lanolin and no lanolin derivatives. The preparation comprises a lipid phase which is characterised in that it includes a mixture of an ester, obtained from the reaction of a long-chain fatty acid with a long-chain fatty alcohol in combination with a liquid polybutene of a molecular weight of between 1500 and 4500 Daltons, preferably between 2200 and 3200 Daltons, and of a viscosity of between 3500 and 5000 mPa·s at 100° C. or greater than 18000 mPa·s at 40° C. respectively.

### DETAILED DESCRIPTION

It was surprisingly found that the combination of a long-chain ester and a fluid polybutene provides a composition which can be well applied and adheres well to the place of application without migrating into other areas of the skin or being transferred on to articles which come into contact therewith. The combination of long-chain ester and fluid polybutene is the essential component of the preparation according to the invention. In addition the preparation may also contain ordinary constituents in a per se known amount, in which respect however there is no need to use waxes, lanolin and lanolin derivatives to achieve the desired properties.

The one important component of the preparation according to the invention is an ester which was obtained by the reaction of a long-chain fatty acid and a long-chain fatty alcohol. In that respect the long-chain fatty acid is a straight-chain, saturated or singly or multiply unsaturated fatty acid with a chain length of between 14 and 36 carbon atoms, preferably between 18 and 26 carbon atoms. The long-chain fatty alcohol used is a straight-chain, saturated or singly or multiply unsaturated alcohol with a chain length of between 16 and 36 carbon atoms, preferably between 18 and 26 carbon atoms. Examples of esters which are suitable according to the invention are for example the esters of palmitic acid, palmitoleic acid, stearic acid, oleic acid, gadoleic acid, behenic acid, erucic acid, eicosanic acid, eicosenic acid, linoleic acid, nervonic acid, or melissinic acid with oleyl alcohol, behenyl alcohol, lignoceryl alcohol, ceryl alcohol, or myricyl alcohol. Buxus chinensis (jojoba oil) or the commercially available synthetic substitutes thereof are preferably used.

The long-chain ester is used in a proportion of between 10 and 65% by weight with respect to the weight of the preparation, preferably between 20 and 45% by weight.

To improve the internal structure a hydrogenated vegetable oil, for example hydrogenated cotton seed oil, hydrogenated castor oil, hydrogenated vegetable oil, hydrogenated rapeseed oil or hydrogenated coco-glycerides are added, and this is also preferred. In addition, to increase viscosity and to provide stabilisation over a wider temperature range, silica (amorphous silicic acid), bentonite, hectorite, montmorillonite or the like can be added. In addition, additives which are usual in cosmetics such as preserving agents, anti-oxidants, aromatic substances, vitamins, sunscreen filters and the like can also be added. Those agents are used in the amounts which are usually known. Examples of preserving agents are parabenes such as methylparabene and propylparabene. Examples of antioxidants and vitamins respectively are tocopherol and ascorbyl palmitate.

The other constituent which is essential according to the invention is a fluid polybutene. The polybutene used can be the per se known polymers which are of a molecular weight in such a range that they are capable of flow. That is usually the case with a molecular weight of between 1500 and 4000 Daltons. Preferably polybutenes of a molecular weight of between 2200 and 3200 Daltons are used. So that the preparation is of the viscosity suitable for application thereof, that value should be between 3500 and 5000 mPa·s at 100° C. or more than 18000 mPa·s at 40° C. The viscosity is determined in per se known manner, for example with a plate/plate measuring system involving a plate diameter of 2 cm and a plate spacing of 400 μm at a shearing speed gradient of 628 s⁻¹.

The content of long-chain esters and polybutene is in each case between 10 and 65% by weight, preferably between 20 and 45% by weight, if these involve coloring agent-bearing formulations, in which respect the quantitative ratio relative to each other is between 2:1 and 1:2. The content of hydrogenated vegetable oil, if present, is in a range of between 0.5 and 10% by weight, preferably between 1.5 and 5.5% by weight. The content of stabilisation agent such as for example silica, bentonite, hectorite, montmorillonite and the like, if present, is generally in a range of between 0.3 and 8% by weight, preferably in a range of between 1 and 5% by weight.

The above-mentioned solid phase can comprise fillers such as for example talcum, kaolin, starch and modified starch, polytetrafluoroethylene powder (Teflon), nylon powder, boronitride, insoluble metal salts such as Mg stearate, Ca stearate, Sr stearate, Zn stearate and inorganic or organic pigments. The following may be mentioned by way of example of the latter: titanium dioxide, zinc oxide, iron oxides, chromium oxide, chromium hydroxide, ultramarine, Berlin blue (ferric blue), mica, pearl gloss agents such as for example mica coated with titanium dioxide, colored mica coated with titanium dioxide and metal oxides, bismuth oxide chloride, coated bismuth oxide chloride, metal powder in flake form of aluminum, brass, bronze, copper, silver, gold and laking means of organic coloring agents with aluminum, barium, calcium or strontium. That list is only given by way of example and is not definitive. Those additives are implemented with the proviso that they are also approved by the respective national or regional cosmetic legislation. Also the amounts used are within the limits of the highest amounts permitted by the respective cosmetic legislation. In that respect the quantitative proportions of pigments are in a range of between 0 and 25% by weight, preferably in a range of between 5 and 20% by weight and quite particularly preferably in a range of between 8 and 15% by weight.

The subject of the invention is further a lipid-bearing preparation which is free of liquid triglycerides. Preferably exclusively solid, hydrogenated glycerides with a melting range of between 30 and 90° C. are used. The following are mentioned by way of example here: hydrogenated cottonseed oil, hydrogenated coco-glycerides, hydrogenated castor oil, hydrogenated rapeseed oil and hydrogenated vegetable oil. The raw materials are identified using the ‘INCI Names’ with which the man skilled in the relevant art is familiar. If hydrogenated triglycerides, in particular hydrogenated vegetable oils, are used, they are preferably added in a proportion of between 0.5 and 10% by weight, particularly preferably between 1.5 and 5.5% by weight, with respect to the weight of the preparation.

It is possible to forego the use of natural or synthetic waxes in the preparations according to the invention, as well as the use of lanolin or lanolin derivatives. In that respect, the term waxes is used to denote vegetable, animal and/or synthetic waxes and/or substitutes thereof, which possibly have a dropping point of between 40 and 130° C. During the storage time in the preparation waxes can form crystallites and minimal crystal structures which can then alter the viscosity—admittedly reversibly but also in such a way as to be noticeable to the consumer. Because of their at least partly pronounced self-emulsifying properties, lanolin and lanolin derivatives, together with the moisture of the skin, can form spontaneous W/O emulsions which can negatively influence adhesion and durability on the skin.

In a preferred embodiment the lipid-bearing preparation is water-free and is in the form of a workable paste. That water-free workable paste preferably is of a dynamic viscosity in the range of between 1.5 and 12 mPa·s, wherein the dynamic viscosity can be determined as described above.

The invention concerns workable lipid-bearing preparations which are applied to the skin, the semi-mucous membranes or in the proximity of mucous membranes, for example in the proximity of the eyes. Here in particular the lips are to be interpreted as semi-mucous membranes. Mention may be made here by way of example of preparations for coloring or for caring for lips, for enhancing shine, in particular lip gloss, for fixing lipstick, preparations for coloring or caring for the skin such as for example make up, blusher, camouflage for hiding age spots or rosacea, concealer and the like, also skin protection products with different sun protection factors (SPF), as far as so-called sun blocks, using very finely divided nanopigments or brightly colored, more highly pigmented sun blocks, which are popular with surfers and windsurfers as body paint.

The preparation can also be produced without the addition of coloring agents and may optionally contain so-called cosmetic active substances. It is then used as lip gloss or as a fixing agent, which are applied over a lipstick. If that uncolored preparation contains light protection filter, it can be used as lip protection and lip care. As is known in contrast to the skin of the body the skin of the lips does not contain any pigmentation. Suitable oil-soluble light filter substances which afford good protection in the UV-A and UV-B range are known in adequate numbers to the man skilled in the relevant art and are regulated by the respective national and regional legislation for example in the EU, in Japan and in the USA—in Germany for example by Appendix 7 to Regulation 3b of the Cosmetics Regulations and they are therefore not to be comprehensively listed here. Therefore mention will only be made by way of example of isoamyl p-methoxycinnamate as a UV-B filter and 4-methylbenzylidene camphor as a UV-A filter.

The preparation according to the invention is in the form of a soft workable paste which can be easily and uniformly applied and distributed. It forms a layer which has a pleasant feel and which has good adhesion, on the above-mentioned regions of the skin, which does not dry and which can remain there the whole day. It can be removed again from the skin in a manner known to the users—by suitable make up removal agents or cloths or by washing with fine soap or suitable mild tenside preparations. It can be filled in known manner into suitable vessels such as bottles, possibly with a spatula, pots or tubes, and can be removed again therefrom by the user. However, because of the improved hygiene conditions involved therewith, it can also be introduced into suitable applicator devices, so-called dispenser mechanisms, and applied therefrom. Applicator devices as are known for example from U.S. Pat. No. 6,238,117 or U.S. Pat. No. 6,309,128 present themselves for the application of small amounts as are required for example for application in the region of lips or eyes, as those devices permit very nice fine metering.

The preparation according to the invention is of such a consistency that it can be very easily introduced for filling purposes into reclosable bottles, pots or tubes. It is particularly suitable for use in a so-called dispenser mechanism. The subject of the invention is therefore also the use of the lipid-bearing preparation according to the invention for filling reclosable bottles, pots or tubes and in particular as a filling for a so-called dispenser mechanism.

The preparation according to the invention will now be described in detail by means of the Examples hereinafter, which however do not definitively describe the invention. In this respect all amounts are stated in percent by weight (% by weight) with respect to the total weight of the preparation:

Example 1 - Pasty lip rouge

| | |
|---|---|
| Buxus chinensis | 39.500 |
| Polybutene | 33.300 |
| Pigments | 18.000 |
| Hydrogenated cottonseed oil | 4.000 |
| Silica | 3.500 |
| Tocopherol | 0.850 |
| Fragrance | 0.300 |
| Methylparabene | 0.200 |
| Propylparabene | 0.150 |
| Ascorbyl palmitate | 0.200 |

Production is effected by a procedure whereby Buxus chinensis, polybutene and hydrogenated cottonseed oil are put in a suitable homogenising machine with an anchor-type agitator and gear ring homogeniser and heated to about 80° C. The silica is then sprinkled in and dispersed by means of the homogeniser. The pigments are then added, and the mixture is then homogenised under a high input of shearing force in order to destroy all pigment agglomerates. The mass is then deaerated by the application of vacuum. Then the preserving agents and anti-oxidants are added to the mixture while still hot and it is then subjected to a brief post-homogenisation operation. The mixture is then cooled down to about 40-50° C. with good agitation with the agitator mechanism. The tocopherol and the fragrance mixture are then added at that temperature. Then, further cooling to about 35° C. is effected with agitation with the agitator mechanism. The mixture which is now pasty is transferred into the filling vessel and allowed to cool down to ambient temperature, without further measures. Then it is filled into the appropriate vessels on a filling or fitting machine, after the preparation has been checked by quality assurance and passed. That gives a workable soft paste of a viscosity of 4500 mPa·s.

Example 2 - Cream eyeshadow

| | |
|---|---|
| Buxus chinensis | 45.000 |
| Polybutene | 27.750 |
| Pigments | 9.000 |
| Mica (and) titanium dioxide | 10.000 |
| Hydrogenated cottonseed oil | 4.000 |
| Silica | 3.000 |
| Tocopherol | 0.500 |
| Fragrance | 0.200 |
| Methylparabene | 0.200 |
| Propylparabene | 0.150 |
| Ascorbyl palmitate | 0.200 |

Manufacture is effected in a similar manner to Example 1, but in this case firstly the coloring agents are added to the fat phase at about 80° C. and homogeneously worked into same, thereafter the pearl gloss agents are added and it is then briefly homogenised once again. Thereafter cooling is effected with good agitation and the fragrance mixture and the tocopherol are added at between 45 and 50° C. The further procedure is then as described above. This gives a soft workable paste with a nice pearl gloss and a viscosity of 3600 mPa·s.

Example 3 - Lipgloss

| | |
|---|---|
| Buxus chinensis | 41.000 |
| Polybutene | 47.800 |
| Hydrogenated cottonseed oil | 4.000 |
| Silica | 2.500 |
| Isoamyl p-methoxycinnamate | 2.000 |
| 4-Methylbenzylidene camphor | 1.000 |
| Tocopherol | 0.900 |
| Fragrance | 0.250 |
| Methylparabene | 0.200 |
| Propylparabene | 0.150 |
| Ascorbyl palmitate | 0.200 |

Manufacture is effected similarly to the above-described processes, wherein the light filter substances are added together with tocopherol and fragrance mixture at about 45-50° C. The result obtained is an uncolored, transparent, very soft paste, with a viscosity of 2400 mPa·s.

Example 4 - Sun block for surfers

| | |
|---|---|
| Buxus chinensis | 40.000 |
| Polybutene | 29.500 |
| Titanium dioxide (nanopigment) | 10.000 |
| Iron oxides (red and yellow) | 5.000 |
| Hydrogenated cottonseed oil | 4.000 |
| Silica | 3.500 |
| Isoamyl p-methoxycinnamate | 3.500 |
| 4-Methylbenzylidene camphor | 2.500 |
| Tocopherol | 1.200 |
| Fragrance | 0.250 |
| Methylparabene | 0.200 |
| Propylparabene | 0.150 |
| Ascorbyl palmitate | 0.200 |

Manufacture is effected in accordance with the process set forth in Example 1. The result obtained is a red-orange soft paste of a viscosity of 3800 mPa·s. The sun protection factor (SPF) of that preparation is above 25.

1-31. (canceled)

32. A cosmetic preparation comprises a lipid phase comprising a mixture of an ester and a liquid polybutene.

33. A cosmetic preparation according to claim 32, wherein the ester is a straight long-chain ester which comprises a reaction product of a long-chain fatty acid with a long-chain fatty alcohol.

34. A cosmetic preparation according to claim 33, wherein the long-chain fatty acid has a chain length of between C-14 and C-36 and the long-chain fatty alcohol has a chain length of between C-16 and C-36.

35. A cosmetic preparation according to claim 32, wherein the straight long-chain ester is Buxus chinensis (jojoba oil).

36. A cosmetic preparation according to claim 35, wherein the polybutene has a molecular weight of between 1500 and 4500 Daltons viscosity between 3500 and 5000 mPa·s at 100° C.

37. A cosmetic preparation according to claim 35, wherein the mixture further comprises a hydrogenated vegetable oil.

38. A cosmetic preparation according to claim 35, wherein the mixture further comprises at least one of a stabilisation agent, a coloring agent and a light protection agent.

39. A cosmetic preparation according to claim 35, wherein the polybutene has a molecular weight of between 2200 and 3200 Daltons viscosity between 3500 and 5000 mPa·s at 100° C.

40. A cosmetic preparation according to claim 38, wherein the viscosity is greater than 18000 mPa·s at 40° C.

41. A cosmetic preparation according to claim 32, wherein the mixture further comprises at least one of fillers, preserving agents, anti-oxidants and fragrances.

42. A cosmetic preparation according to claim 32, wherein the mixture is free of vegetable, animal or synthetic waxes and/or substitutes thereof with a dropping point of between 40° C. and 130° C. and/or lanolin and derivatives thereof.

43. A cosmetic preparation according to claim 32, wherein the cosmetic preparation comprises a decorative cosmetic for care of, coloring and improving the appearance of the skin, the lips and the eyelids.

44. A cosmetic preparation according to claim 32, wherein the cosmetic preparation is a rouge, lip rouge, make-up, camouflage or a concealer.

45. A cosmetic preparation according to claim 32, wherein the cosmetic preparation is in the form of lip care, a fixing agent for the lips, a care foundation, an agent for caring for the skin or a sun protection agent.

46. A cosmetic preparation according to claim 32, wherein the mixture is water-free.

47. A cosmetic preparation according to claim 46, wherein the mixture is a water-free workable paste.

48. A cosmetic preparation according to claim 47, wherein the water-free workable paste has a dynamic viscosity in a range of between 1.5 and 12 Pas.

49. A cosmetic preparation according to claim 48, wherein the dynamic viscosity of the water-free workable paste is measured with a plate/plate measuring system with a plate diameter of 2 cm and a plate spacing of 400 μm at a shearing speed gradient of 628 1/s.

50. A cosmetic preparation according to claim 33, wherein the content of straight long-chain ester and liquid polybutene is between 10% by weight and 65% by weight, respectively, and wherein the quantitative ratio with each other is between 2:1 and 1:2.

51. A cosmetic preparation according to claim 33, wherein the content of straight long-chain ester and liquid polybutene is between 20% by weight and 45% by weight, respectively, and wherein the quantitative ratio with each other is between 2:1 and 1:2.

52. A cosmetic preparation according to claim 32, wherein the mixture further comprises solid hydrogenated triglycerides having a melting range of between 30° C. and 90° C.

53. A cosmetic preparation according to claim 52, wherein the solid hydrogenated triglycerides are hydrogenated vegetable oils.

54. A cosmetic preparation according to claim 53, wherein the content of hydrogenated vegetable oil is in a range of between 0.5 and 10% by weight.

55. A cosmetic preparation according to claim 53, wherein the content of hydrogenated vegetable oil is in a range of between 1.5 and 5.5% by weight.

56. A cosmetic preparation according to claim 32 further comprising at least one of silica, bentonite, hectorite, montmorillonite and mixtures thereof as a stabilising agent.

57. A cosmetic preparation according to claim 56, wherein the content of the stabilizing agent is between 0.3 and 8% by weight.

58. A cosmetic preparation according to claim 56, wherein the content of the stabilizing agent is between 1 and 5% by weight.

59. A cosmetic preparation according to claim 32, further comprising a solid phase comprising at least one of fillers, inorganic pigments, organic pigments, and mixtures thereof.

60. A cosmetic preparation according to claim 59, wherein the fillers are selected from the group consisting of talcum, kaolin, starch, modified starch, polytetrafluoroethylene powder, nylon powder, boronitride, Mg stearate, Ca stearate, Sr stearate, Zn stearate and mixtures thereof.

61. A cosmetic preparation according to claim 59, wherein the inorganic pigments are selected from the group consisting of titanium dioxide, zinc oxide, iron oxides, chromium oxide, chromium hydroxide, ultramarine, Berlin Blue (ferric blue), mica, mica coated with titanium dioxide, mica coated with titanium dioxide and with metal oxides, bismuth oxide chloride, coated bismuth oxide chloride, metal powder in flake form of aluminum, brass, bronze, copper, silver, gold and mixtures thereof.

62. A cosmetic preparation according to claim 59, wherein the organic pigments comprise Takings of organic coloring agents with at least one of aluminum, barium, calcium, strontium, and zirconium.

63. A cosmetic preparation according to claim 59, wherein the quantitative proportions of pigments are in a range of between 0.1 and 25% by weight.

64. A cosmetic preparation according to claim 59, wherein the quantitative proportions of pigments are in a range of between 5 and 20% by weight.

65. A cosmetic preparation according to claim 59, wherein the quantitative proportions of pigments are in a range of between 8 and 15% by weight.

66. A cosmetic preparation according to claim 32, wherein the preparation is a light protection agent which comprises at least one of titanium dioxide and zinc oxide in the form of nanopigments having a particle size of between 5 and 25 nm.

67. A cosmetic preparation according to claim 66, wherein the nanopigments further comprise oil-soluble UV-A- and UV-B-light filter substances.

68. A cosmetic preparation according to claim 67, wherein the light protection agent further comprises 4-methylbenzylidene camphor and isoamyl p-methoxycinnamate.
